(19) United States
(12) Patent Application Publication
(10) Pub. No.: US 2019/0231835 A1
(43) Pub. Date: Aug. 1, 2019

(54) PHARMACEUTICAL COMPOSITION CONTAINING SOPHORA JAPONICA L. EXTRACT AS ACTIVE INGREDIENT FOR THE PREVENTION AND TREATMENT OF NEURODEGENERATIVE DISORDERS

(71) Applicant: UNIVERSITY-INDUSTRY COOPERATION GROUP OF KYUNG HEE UNIVERSITY, Yongin-si, Gyeonggi-do (KR)

(72) Inventors: Seung-Hun CHO, Seoul (KR); Hwa-Young LEE, Incheon (KR); Yongju KWON, Seoul (KR)

(21) Appl. No.: 16/328,194

(22) PCT Filed: Aug. 25, 2016
(86) PCT No.: PCT/KR2016/009448

§ 371 Date: Feb. 25, 2019

(51) Int. Cl. A61K 36/489; A61P 25/28; A23L 33/105

(52) U.S. Cl. CPC A61K 36/489; A61P 25/28; A23V 2250/21; A23V 2002/00; A23V 2200/322; A23L 33/105

(57) ABSTRACT

Provided is a pharmaceutical composition containing, as an active ingredient, a Sophora japonica L. extract, for the prevention and treatment of neurodegenerative disorders and improvement of cognitive functions. Particularly, it was confirmed that the Sophora japonica L. extract, according to the present invention, has a significant effect in an Alzheimer's Disease-causing mouse model, through a Y-maze test and a Morris water maze test, thereby confirming that the Sophora japonica L. extract could be useful as an active ingredient in the pharmaceutical composition for the prevention and treatment of neurodegenerative disorders and the improvement of cognitive functions.

### TECHNICAL FIELD

The present invention relates to pharmaceutical compositions for preventing and treating a neurodegenerative disorder and improving a cognitive function, each of which includes a Sophora japonica L. extract as an active ingredient.

### BACKGROUND ART

Alzheimer's disease is a disease that leads to an enormous burden on the family and the society as well as a dementia patient. Entering the aging society, along with the interest in aging, the interest in cerebral nervous diseases such as aging-related diseases, stroke, and Alzheimer's dementia are increasing. Among the various brain diseases, dementia is a disease causing the most widespread cell damage, and accompanying degenerative metal disorders, and particularly, major symptoms such as memory impairment and loss of judgment are well known. Dementia has various causes, and may be largely divided into vascular dementia (20%˜30%) caused by stenosis or occlusion of cerebral vessels, Alzheimer's dementia (50%) known to occur due to accumulation of β-amyloid proteins in the brain, and mixed-type dementia (15%˜20%) caused by a combination of these two types. The type of dementia that accounts for the largest percentage of patients is Alzheimer's dementia, and according to a recent study, one out of 85 people will have the disease by 2050, and 43% of the patients will need intensive care (Prabhulkar S, Piatyszek R, et al. J Neurochem., 2012, 122, 374-381).

Alzheimer's disease is largely classified into familial Alzheimer's disease (FAD) and sporadic Alzheimer's disease (SAD). FAD occurs in approximately 5% to 10% of the total patients with Alzheimer's disease, and when mutations occur in presenilin 1 (PS1), amyloid precursor protein (APP) and presenilin 2 (PS2), known as causative genetic factors, 100% of the patients have Alzheimer's disease. SAD accounts for almost all of the patients with Alzheimer's disease, and when mutations occur in apolipoprotein E (ApoE) or α-2 macroglobulin (A2M), risk factors that increase the probability of developing Alzheimer's disease have been found. However, the exact cause of the onset is not yet known.

Pathological characteristics of Alzheimer's disease may include senile plaques accumulated outside of nerve cells, neurofibrilary tangles appearing like a bundle of threads tangled in the cell body of a nerve cell, and neuronal loss. These pathological characteristics are shown in both cases of FAD and SAD, and among these, a toxic protein called aggregated amyloid beta peptide (Aβ) is known as a major component of senile plaques. The amyloid beta peptide is an insoluble peptide consisting of 40 to 42 amino acids produced by abnormal cleavage of the amyloid precursor protein. In addition, it has been reported that excessive accumulation of the amyloid beta peptide is the common phenomenon occurring in both cases of FAD and SAD. Therefore, the amyloid beta peptide is considered as the main pathogenic material of Alzheimer's disease. In the overall process of Alzheimer's disease, when mutation of presenilin 1 and 2 genes (PS1 and PS2) occurs, the amyloid precursor protein is abnormally cleaved by β-secretase, and the amyloid beta peptide is produced. It has been known that necrosis of brain nerve cells occurs due to the produced amyloid beta peptide, and thereby Alzheimer's disease occurs.

To date, a variety of studies have been conducted on the etiology and a treatment method of dementia, but the cause of the disease has not been identified and an effective treatment method has not yet been developed. Although tacrine, rivastigmine, galantamine, donepezil and memantine were approved as therapeutic agents for Alzheimer's disease from the Food and Drug Administration (FDA), most currently used dementia drugs are merely materials for alleviating mental degeneration, which alleviate symptoms of degenerative dementia, and most of the drugs are anti-inflammatory drugs having side effects such as hepatotoxicity and mucosal damage in a digestive organ, and they are limited to symptomatic therapy, rather than treatment of an ultimate cause. An acetylcholine neurotransmitter is a drug which induces enhancement of a brain cognitive function, and only temporarily relieves the progression or symptoms of dementia. Furthermore, as the death of nerve cells progresses, a drug effect is reduced, and in the case of severe dementia, there is no drug effect. In addition, most drugs for Alzheimer's disease which have been studied to date use an ion channel blocker such as a glutamic acid receptor blocker, an antioxidant, calcium or sodium, and effective drugs have not yet been developed. Therefore, it is required to transition to innovative ideas and discover a new concept of novel therapeutic agents.

Sophora japonica L. is a fruit of the Chinese scholar tree which is a deciduous tree belonging to the pea family. Sophora japonica L. is native to Korea and China, is distributed all over the countries, and is used for ornamental, industrial, edible and medicinal purposes. In non-official and oriental medicine, Sophora japonica L. is a tree with excellent efficacy in treating inflammation, hemostasis, hypertension, hemorrhoids and eczema, and has been known from ancient times as a tree with excellent properties as a medicine for brightened eyes, avoidance of whitened beard and hair, and a long life. The flower and fruit of Sophora japonica L. are called goesil, goeja, goedu, goewha, and Sophora japonica L., and the main components of Sophora japonica L. are 9 flavonoid and isoflavonoid compounds including sophoraflavonoloside, genistein, sophorabioside, kaempferol, rutin, and glucoside-C. It has been known that a rutin content in the young fruit of Sophora japonica L. reaches 1.76%, and Sophora japonica L. has been known to be effective in improvement of hyperlipidemia, antioxidation, anti-anxiety, and improvement in menopausal syndrome.

Meanwhile, as prior art relating to Sophora japonica L., it has been known that a methanol extract of Sophora japonica L. exhibits an antianxiety action (Jeong, Ji Wook et al., Korean J. Food Preservation., 2012, 19(5), 767-773), in Korean Unexamined Patent Application No. 2005-0089182, the anticancer effect of the Sophora japonica L. extract is disclosed, and in Korean Unexamined Patent Application No. 2005-0050728, a therapeutic effect of the Sophora japonica L. extract on a metabolic bone disease is disclosed, but the effect of the Sophora japonica L. extract on neurodegenerative disorders including Alzheimer's disease is not yet known.

Therefore, the inventors have attempted to develop a therapeutic agent for a neurodegenerative disorder using a natural substance with less side effects, and confirmed that the Sophora japonica L. extract of the present invention exhibits significant dementia and cognitive function improvement effects in Alzheimer's disease-induced animal models, demonstrating that the Sophora japonica L. extract can be effectively used as an active ingredient of a pharmaceutical composition for preventing and treating a neurodegenerative disorder and a composition for improving a cognitive function. Therefore, the present invention was completed.

### DISCLOSURE

### Technical Problem

The present invention is directed to providing pharmaceutical compositions for preventing and treating a neurodegenerative disorder and improving a cognitive function, each of which contains a Sophora japonica L. extract.

### Technical Solution

In one aspect, the present invention provides a pharmaceutical composition for preventing and treating a neurodegenerative disorder, which includes a Sophora japonica L. extract as an active ingredient.

In another aspect, the present invention provides a health functional food composition for preventing and alleviating a neurodegenerative disorder, which includes a Sophora japonica L. extract as an active ingredient.

In still another aspect, the present invention provides a pharmaceutical composition for improving a cognitive function, which includes a Sophora japonica L. extract as an active ingredient.

In yet another aspect, the present invention provides a health functional food composition for improving a cognitive function, which includes a Sophora japonica L. extract as an active ingredient.

### Advantageous Effects

As it is confirmed through a Y-maze test and a Morris water maze test for Alzheimer's disease-induced mouse models that a Sophora japonica L. extract of the present invention has a significant effect, the Sophora japonica L. extract can be used as an active ingredient in pharmaceutical compositions for preventing and treating a neurodegenerative disorder and improving a cognitive function.

### DESCRIPTION OF DRAWINGS

FIG. 1 is a graph showing a walking distance according to administration of a Sophora japonica L. extract to an Alzheimer's disease-induced mouse model:

Distance average (Dist average): a walking distance of a mouse;

Sham control (SC): a mouse group into which the amyloid beta peptide is not infused;

Negative control (NC): a mouse group which is treated with the amyloid beta peptide, but not treated with donepezil;

Positive control (PC): a mouse group which is treated with the amyloid beta peptide and donepezil;

Experiment 1 (Exp. 1): a mouse group which is treated with the amyloid beta peptide, and 100 mg/kg of the Sophora japonica L. extract of the present invention;

Experiment 2 (Exp. 2): a mouse group which is treated with the amyloid beta peptide, and 600 mg/kg of the Sophora japonica L. extract of the present invention.

FIG. 2 is a graph showing a cognitive function improvement effect caused by administration of a Sophora japonica L. extract to an Alzheimer's disease-induced mouse model.

FIG. 3 is a graph showing a learning and memory improvement effect caused by administration of a Sophora japonica L. extract to an Alzheimer's disease-induced mouse model.

### MODES OF THE INVENTION

Hereinafter, the present invention will be described in detail.

The present invention provides a pharmaceutical composition for preventing and treating a neurodegenerative disorder, which includes a Sophora japonica L. extract as an active ingredient.

The Sophora japonica L. extract may be prepared by a preparation method including following steps, but the present invention is not limited thereto:

1) performing extraction by treating Sophora japonica L. with an extraction solvent;

2) filtering the extract of Step 1); and

3) preparing an extract of Sophora japonica L. by vacuum-evaporating and then drying the filtered extract of Step 2).

In this method, the Sophora japonica L. of Step 1) may be one which is grown or commercially available without limitation.

In the method, the extraction solvent of Step 1) may be water, an alcohol or a mixture thereof, and an organic solvent. As the alcohol, a C₁ to C₂ lower alcohol may be used, and as a lower alcohol, ethanol or methanol may be used. As an extraction method, shaking culture, Soxhlet extraction or reflux culture may be used, but the present invention is not limited thereto. Extraction is preferably performed by adding the extraction solvent at an amount 1 to 10-fold higher than the amount of dried Sophora japonica L., and more preferably by adding the extraction solvent at an amount 4 to 6-fold higher than the amount of dried Sophora japonica L. An extraction temperature is preferably 20 to 100° C., and more preferably 20 to 40° C., and even more preferably room temperature, but the present invention is not limited thereto. In addition, an extraction time is preferably 10 to 48 hours, more preferably 15 to 30 hours, and even more preferably 24 hours, but the present invention is not limited thereto. In addition, the number of times of extraction is preferably 1 to 5 times, more preferably 3 to 4 times, and even more preferably 3 times, but the present invention is not limited thereto.

The obtained Sophora japonica L. extract may be stored in a deep freezer until use.

The neurodegenerative disorder may be any one selected from the group consisting of dementia, Alzheimer's disease, stroke, palsy, Huntington's disease, Pick's disease, and Creutzfeldt-Jakob disease, but the present invention is not limited thereto.

In a specific exemplary embodiment of the present invention, the inventors prepared a Sophora japonica L. extract, and then performed a Morris water maze test, to confirm a cognitive disorder improvement effect of the Sophora japonica L. extract, after the Sophora japonica L. extract was administered into an Alzheimer's disease-induced mouse model in which the amyloid beta peptide (amyloid β₁₋₄₂ peptide) was infused into the brain, and therefore, it was confirmed that learning and memory loss caused when Alzheimer's disease occurs can be alleviated, demonstrating that the Sophora japonica L. extract has an effect of improving and treating Alzheimer's dementia (see FIG. 3).

Therefore, the Sophora japonica L. extract of the present invention can be used as a pharmaceutical composition for preventing and treating a neurodegenerative disorder due to an effect of alleviating Alzheimer's disease.

A composition containing the Sophora japonica L. extract of the present invention may further contain one or more active ingredients exhibiting the equal or similar function to the above-described ingredient, in addition to the above-described ingredient.

The composition of the present invention may further include a pharmaceutically acceptable additive, and as the pharmaceutically acceptable additive, starch, gelatinized starch, microcrystalline cellulose, lactose, povidone, colloidal silicon dioxide, calcium hydrogen phosphate, lactose, mannitol, crude maltose, gum arabic, pregelatinized starch, corn starch, powdered cellulose, hydroxypropylcellulose, Opadry, sodium starch glycolate, carnauba wax, synthetic aluminum silicate, stearic acid, magnesium stearate, aluminum stearate, calcium stearate, white sugar, dextrose, sorbitol and talc may be used. The pharmaceutically acceptable additive according to the present invention may be included at 0.1 to 90 parts by weight with respect to the composition, but the present invention is not limited thereto.

In other words, the composition of the present invention may be administered in various formulations including oral and non-oral formulations when clinically administered, and in preparation, the composition of the present invention may be formulated using a diluent or an excipient such as a filler, a thickening agent, a binder, a wetting agent, a disintegrant, a surfactant, which are conventionally used. A solid formulation for oral administration may be a tablet, pill, powder, granule or capsule, and such a solid formulation may be prepared by mixing at least one excipient, for example, starch, calcium carbonate, sucrose, lactose and gelatin, with the active ingredient. Also, in addition to the simple excipient, lubricants such as magnesium stearate and talc may also be used. As a liquid formulation for oral administration, a suspension, a liquid for internal use, an emulsion, or a syrup may be used, and a generally-used simple diluent such as water or liquid paraffin, as well as various types of excipients, for example, a wetting agent, a sweetener, a fragrance and a preservative may be included. A formulation for parenteral administration includes a sterilized aqueous solution, a non-aqueous solvent, a suspension, an emulsion, a lyophilizing agent and a suppository. As the non-aqueous solvent or suspension, propylene glycol, polyethylene glycol, a vegetable oil such as olive oil, or an injectable ester such as ethyl oleate may be used. As a suppository base, Witepsol, Tween 61, cacao butter, laurin fat, or glycerogelatin may be used.

The composition of the present invention may be administered orally or non-orally according to a desired method, and for non-oral administration, an external use for skin, or intraperitoneal injection, intrarectal injection, subcutaneous injection, intravenous injection, intramuscular injection or intrathoracic injection may be selected. A dose may vary according to a patient's body weight, age, sex or health condition, diet, an administration time, an administration method, an excretion rate and the severity of a disease.

The dose of the composition of the present invention varies depending on a patient's body weight, age, sex or health condition, diet, an administration time, an administration method, an excretion rate or the severity of a disease, and a daily dose may be 0.0001 to 100 mg/kg, and preferably, 0.001 to 10 mg/kg based on the amount of the Sophora japonica L. extract, and administered 1 to 6 times a day.

The composition of the present invention may be used alone, or in combination with a surgery, radiation therapy, hormone therapy, chemotherapy or a method using a biological response modifier.

IN addition, the present invention provides a health functional food composition for preventing and alleviating a neurodegenerative disorder, which includes a Sophora japonica L. extract as an active ingredient.

It was confirmed that the Sophora japonica L. extract of the present invention diminishes learning and memory loss, which can occur when Alzheimer's disease is caused, and thus can be used as a health functional food composition for preventing and alleviating a neurodegenerative disorder.

The “health functional food” used herein is prepared using nutrients that are likely to be deficient in daily meals or raw materials or ingredients with a function useful for the human body (functional raw materials), means a food that maintains a normal function of the human body or maintains and improves health through the activation of a physiological function, is notified by the minister of the Ministry of Food and Drug Safety (MFDS), but the present invention is not limited thereto. It is not meant to exclude healthy food in its usual acceptation.

The Sophora japonica L. extract of the present invention may be added directly to food or in combination with another food or food ingredient, and may be suitably used by a conventional method. A mixing amount of the active ingredient may be suitably determined according to the purpose of use (for prevention or improvement). Generally, an amount of the compound in the health functional food may be applied at 0.01 to 90 parts by weight of the total food weight. However, in the case of long-term ingestion for health and hygiene or health control, the amount may be less than the above range, and since there is no problem in terms of safety, the active ingredient may be used at an amount more than the above range.

A health functional drink composition of the present invention may contain various favoring agents or natural carbohydrates as additional components, like a conventional drink, in addition to the Sophora japonica L. as an essential component at the above-mentioned proportion. The above-mentioned natural carbohydrate may be a monosaccharide such as glucose or fructose; a disaccharide such as maltose or sucrose; a polysaccharide such as dextrin or cyclodextrin; or a sugar alcohol such as xylitol, sorbitol or erythritol. As a sweetening agent, a natural sweetening agent such as a thaumatin or stevia extract; or a synthetic sweetening agent such as saccharin or aspartame may be used. A ratio of the natural carbohydrate may be generally approximately 1 to 20 g, and preferably approximately 5 to 12 g per 100 g of the composition of the present invention.

Other than the above ingredients, the health functional drink composition of the present invention may contain various nutrients, vitamins, minerals (electrolytes), flavoring agents such as synthetic and natural flavoring agents, colorants, enhancers (cheese, chocolate, etc.), pectic acid or a salt thereof, alginic acid or a salt thereof, organic acids, protective colloidal thickening agents, pH adjusters, stabilizers, preservatives, glycerin, an alcohol or a carbonating agent used in a carbonated drink. In addition, the health functional drink composition of the present invention may contain fruit flesh for producing a natural fruit juice, a fruit juice drink, or a vegetable drink.

Such ingredients may be used independently or in combination thereof. A ratio of the additive is not important, but generally selected in a range of 0.1 to approximately 20 parts by weight with respect to 100 parts by weight of the Sophora japonica L. extract of the present invention.

In addition, the present invention provides a pharmaceutical composition and a health functional food composition for improving a cognitive function, which includes a Sophora japonica L. extract as an active ingredient.

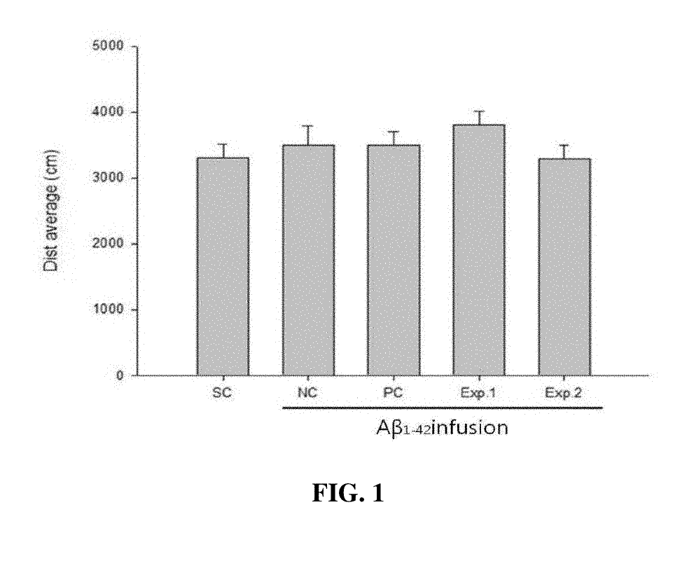

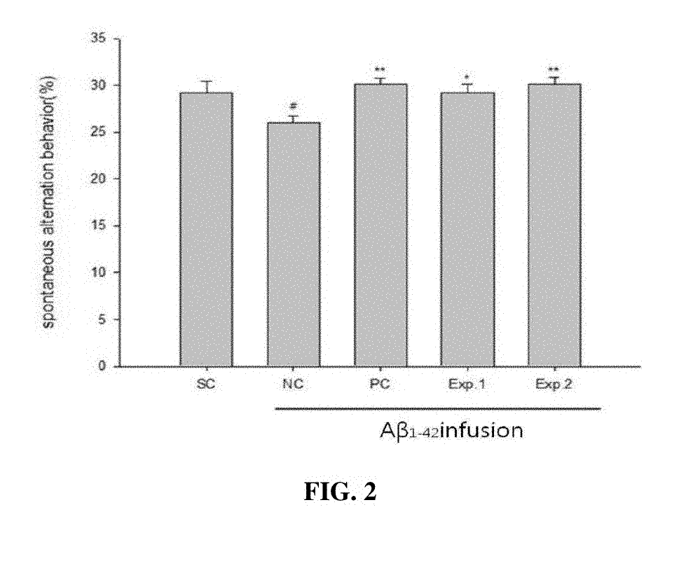

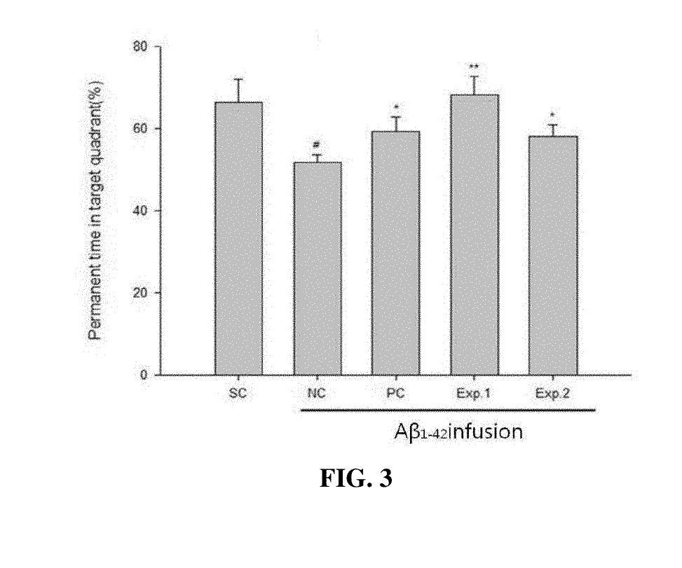

In a specific exemplary embodiment of the present invention, the inventors confirmed that a Sophora japonica L. extract increases spontaneous alternation without influencing a walking distance of a mouse by administering the Sophora japonica L. extract into a prepared Alzheimer's disease-induced mouse model (see FIGS. 1 and 2), demonstrating that the Sophora japonica L. extract can be used as the pharmaceutical composition and health functional food composition for improving a cognitive function, which are effective in alleviation of Alzheimer's disease.

Hereinafter, the present invention will be described in detail with reference to examples and experimental examples.

However, the following examples and experimental examples are merely provided to illustrate the present invention, but the scope of the present invention is not limited to the following examples and experimental examples.

### <Example 1>Preparation of Sophora japonica L. Extract

<1-1>Preparation of Water Extract of Sophora japonica L.

Sophora japonica L. harvested in Yeosu, Jeollanam-do was dried to be used in the present invention. 100 g of pulverized Sophora japonica L. was applied to 1 L of distilled water, well stirred, subjected to reflux extraction at an extraction temperature of 90 to 95° C. for 3 hours, thereby separating a filtrate, and the Sophora japonica L. extract was subjected to vacuum evaporation at 55 to 65° C. and then freeze-drying, thereby obtaining 21.2 g of a water extract powder of Sophora japonica L.

<1-2>Preparation of 30% Alcohol Extract of Sophora japonica L.

According to the same method as described in Example <1-1>, 3 L of 30% ethyl alcohol was added to 550 g of pulverized Sophora japonica L. extract, and the mixture was well stirred and heated to perform reflux extraction at an extraction temperature of 80 to 90° C. for 3 hours, thereby isolating a filtrate, and the Sophora japonica L. extract was subjected to vacuum evaporation at 55 to 65° C. and freeze-drying, thereby obtaining 139.5 g of a 30% alcohol extract powder of Sophora japonica L.

### <Example 2>Preparation of Alzheimer's Disease-Induced Animal Model

To prepare an Alzheimer's disease-induced animal model infused with the amyloid beta peptide (amyloid beta₁₋₄₂ peptide) (amyloid beta₁₋₄₂ peptide-infused mouse model), an experiment was carried out as follows.

Specifically, a C57BL/6 mouse was anesthetized with a 2:1 mixture of Zoletil and Rompun, infused with the amyloid beta peptide in the hippocampus CA1 region in the brain (coordinates: −2.3 mm anterior/posterior, 1.8 mm medial/lateral and −1.75 mm dorsal/ventral from the bregma), thereby preparing an Alzheimer's disease-induced mouse model.

### <Experimental Example 1>Confirmation of Effect of Sophora japonica L. Extract on Walking Distance

To observe whether the Sophora japonica L. extract extracted by the method described in Example 1 affects changes in basic mobility and movement of a mouse, an experiment was carried out as follows.

Specifically, a locomotor activity test was carried out by putting a mouse into a white acrylic box with dimensions of 50 cm×50 cm×50 cm, and monitoring behavior using a video tracking system (Smart program v.2.5.21) for 10 minutes, and the open space was divided into 9 sections, and the central section was set as a central zone.

Alzheimer's disease-induced mouse models prepared in the method described in Example 2 was respectively administered 1 mg/kg of a therapeutic agent for a neurodegenerative disorder, donepezil, 100 or 600 mg/kg of the Sophora japonica L. extract of the present invention, and a control, distilled water, and then a walking distance was measured using spontaneous locomotor activity.

As a result, as shown in FIG. 1, in the case of animal models respectively administered distilled water, the Sophora japonica L. extract, and donepezil after induction of Alzheimer's disease, compared with an animal model in which Alzheimer's disease was not induced, there was no significant difference, indicating that there was no change in walking distance due to drug administration (FIG. 1).

This result showed that there is no problem in exercise function that can be generated by drug administration, confirming that the Sophora japonica L. extract does not affect a walking distance, and the reliability of the following experimental example was able to be confirmed.

### <Experimental Example 2>Confirmation of Effect of Improving Cognitive Disorder by Sophora japonica L. Extract

To evaluate an effect of spontaneous spatial perception in the form of short-term memory of the Sophora japonica L. extract prepared in Example 1, a Y-maze test was carried out using the Alzheimer's disease-induced mouse model prepared as described in Example 2.

Specifically, the apparatus used in the Y-maze test has three arms, each arm having a length of 42 cm, a width of 3 cm and a height of 12 cm, and an angle between the three arms is 120°. All experimental devices are formed of black polyvinyl plastic. The respective arms are set as A, B and C, mice were carefully put at one arm and allowed to freely move for 8 minutes, and then an arm into which a mouse entered was recorded. Here, a mouse was recognized to have entered an arm only when the tail of the mouse completely entered, and an arm which the mouse re-entered was also recorded. When a mouse sequentially entered three different arms (ABC, CAB, BCA; actual alternation), one point was given. Alternation behavior is defined as a mouse sequentially entering all three arms, and was calculated by the following mathematical formula.

Alternation behavior (%)=actual alternation/maximum alternation×100(maximum alternation:the total number of times of entry−2) [Mathematical Formula]

As a result, as shown in FIG. 2, it was confirmed that the alternation behavior of a negative control induced by directly infusing the amyloid beta peptide into the brain (1.2 μg/mouse, i.c.v) was significantly lower than that of a non-infused control (p<0.05). However, spontaneous alternation was significantly increased in a Sophora japonica L. extract-administered group, compared with a negative control (P<0.05). The increase in spontaneous alternation means that learning and memory are restored. On the other hand, since there is no change in the total entry representing the total number of the times of entry into each zone, it was confirmed that spontaneous alternation is not caused by a change in mouse activity (FIG. 2).

### <Example 3>Confirmation of Alzheimer's Disease Alleviation Effect of Sophora japonica L. Extract

To confirm an Alzheimer's disease alleviation effect of the Sophora japonica L. extract of the present invention, a Morris water maze test, which is a passive avoidance test, widely used as a method for measuring spatial learning and memory of a rodent was performed.

Specifically, the following experiment was carried out by applying a conventional Morris method. First, a mouse was transferred to a behavior observation chamber and stabilized one hour before the initiation of the experiment. The maze dimension includes a diameter of 90 cm and a height of 32.5 cm, and the diameter of a white platform is 5 cm. In the periphery of the underwater maze, spatial cues such as a computer system connected with a video camera and a device for controlling a water temperature were always regularly maintained. Afterward, the maze was filled with water so that the platform is installed 1 cm below the water level, such that a mouse cannot see the platform. The maze was divided into quadrants using four markers, the quadrants were classified as northeast (NE), northwest (NW), southeast (SE), and southwest (SW), and the platform was installed in one quadrant of the maze. The Morris water maze test was carried out for 6 days, and on the first day, each mouse is allowed to freely swim in the maze for 1 minute to be adjusted to water, and at this time, the platform was not installed. From the second day to the fifth day, each mouse was allowed to swim in the maze at intervals of 10 minutes for one minute four times a day. In a single cycle of the experiment performed four days from the second day to the fifth day, an experiment for a mouse having ascended the platform previously installed in the maze for 1 second within 1 minute was finished, and an experiment for a mouse, which did not find the platform within 1 minute or did not ascend the platform for 10 seconds, which was artificially placed on the platform for 1 second after the finish of the experiment was finished, and at this time, the platform was fixed at the same position. On the sixth day, the platform was removed from the maze, and then the residence time of a mouse in the quadrant in which the platform was placed was measured. On the final day (day 5) of the Morris water maze test, to perform a memory test, the platform was removed, free swimming was allowed for 60 seconds, and the residence time of a mouse in the quadrant in which the platform was placed was measured in percentage using a Smart program.

As a result, as shown in FIG. 3 and Table 1, according to the memory test for each group, a control (Sham) is 64.274±5.030954%, the amyloid beta peptide-administered group (negative control) is 51.618±1.420522%, the amyloid beta peptide+donepezil (1 mg/kg)-administered group (positive control) is 59.222±2.917772%, the amyloid beta peptide+Sophora japonica L. extract 100 mg/kg-administered group (Exp.1) is 67.428±4.094657%, and the amyloid beta peptide+Sophora japonica L. extract 600 mg/kg-administered group (Exp.2) is 58.75±2.574205%, showing a significant difference between groups (P<0.05) (FIG. 3). This result showed that learning and memory loss, which may occur when Alzheimer's disease is induced, can be diminished by the Sophora japonica L. extract, and therefore the Sophora japonica L. extract is effective in improving and treating Alzheimer's disease.

TABLE 1

| Group | Aβ infusion | Residence time in quadrant (%) |
|---|---|---|
| Control (Sham) | X | 64.274 ± 5.030954 |
| Negative | ◯ | 51.618 ± 1.1420522^{#} |
| Positive | ◯ | 59.222 ± 2.917772* |
| Exp. 1 | ◯ | 67.428 ± 4.094657** |
| Exp. 2 | ◯ | 58.75 ± 2.574205* |

Each value represents an average S.E.M. Compared with the control (Sham), ^{#}p < 0.05, and compared with the Aβ-infused group, *p < 0.05.

1. A pharmaceutical composition for preventing and treating a neurodegenerative disorder, comprising:
a Sophora japonica L. extract as an active ingredient.

2. The pharmaceutical composition according to claim 1, wherein the extract is extracted with water, a C₁ to C₂ lower alcohol or a mixture thereof.

3. The pharmaceutical composition according to claim 2, wherein the C₁ to C₂ lower alcohol is ethanol or methanol.

4. The pharmaceutical composition according to claim 1, wherein the neurodegenerative disorder is any one selected from the group consisting of dementia, Alzheimer's disease, stroke, palsy, Huntington's disease, Pick's disease, and Creutzfeldt-Jakob disease.

5. A health functional food composition for preventing and alleviating a neurodegenerative disorder, comprising:
a Sophora japonica L. extract as an active ingredient.

6. A pharmaceutical composition for improving a cognitive function, comprising:
a Sophora japonica L. extract as an active ingredient.

7. A health functional food composition for improving a cognitive function, comprising:
a Sophora japonica L. extract as an active ingredient.
